(19) United States
(12) Patent Application Publication
(10) Pub. No.: US 2026/0273490 A1
(43) Pub. Date: Sep. 17, 2026

(54) PROCESSING A PRIMARY PRODUCT INTO A SECONDARY PRODUCT

(71) Applicant: BASF SE, Ludwigshafen am Rhein (DE)

(72) Inventors: Hans RUDOLPH, Lemfoerde (DE); Michael HARTMANN, Lemfoerde (DE); Ingo WINDELER, Lemfoerde (DE); Robert PACK, Ludwigshafen am Rhein (DE); Christian-Andreas WINKLER, Ludwigshafen am Rhein (DE)

(21) Appl. No.: 19/164,056

(22) PCT Filed: Mar. 12, 2024
(86) PCT No.: PCT/EP2024/056575

§ 371 Date: Sep. 10, 2025

(30) Foreign Application Priority Data
Mar. 13, 2023 (EP) 23161600.4

(51) Int. Cl. B01J 19/00; G16C 20/90

(52) U.S. Cl. CPC B01J 19/0006; G16C 20/90

(57) ABSTRACT

A method (100) for processing a primary product (A) into a secondary product (C), including the steps: providing (S15) at least one distinguishable quantity of a primary product (A); providing (S11) at least one a data structure (4′) associated with the distinguishable quantity of a primary product (A), said data structure (4′) including one data item (5) holding an object identifier indicative of the distinguishable quantity of the primary product (A); processing (S19) the at least one primary product (A) into a distinguishable quantity of a secondary product (C) by a processing device (11); generating (S28) an unique object identifier indicative of said obtained distinguishable quantity; generating (S29) a new data structure (4) having at least one data item (5) holding the generated object identifier and one data item (5) holding the object identifier indicative of the quantity of the primary product (A); and providing (S52) said quantity obtained during the processing step (S19).

This disclosure relates to a method for processing at least one primary product into at least one secondary product. Further, this method relates to a computer program product, a data structure having at least two data items, and a data carrier signal.

Document US 2022/237628 A1 discloses a workflow for tracking carbon credits. Upon conversion of a primary product (e.g. a polymer) into a secondary product (e.g. a resin), a blockchain entry is created, which includes a packet holding details on the secondary product and a packet holding details on the primary product.

In order to produce advanced products, it is desired to increase a knowledge of properties of these products. It is therefore an object of the present disclosure to provide means for transferring more precisely defined properties of a product to a next processing phase within a product life cycle.

Below, there are presented multiple solutions to this object. However, it seems important to mention that the presented aspects and options can be implemented all together. Thus, any feature disclosed in relation to one aspect and/or option can be combined with every other aspect or option.

According to one aspect of the invention, a method for processing at least one primary product into at least one secondary product is suggested. The method includes providing at least one primary product; processing the at least one primary product into a distinguishable quantity of a secondary product by a processing device; generating a report by a state reporting device associated with the processing device, wherein said report includes at least one state value indicative of a processing state during the processing step; generating a unique object identifier indicative of said distinguishable quantity, which distinguishable quantity is obtained by said processing during the processing step; generating a data structure having at least two data items, wherein one data item holds the object identifier, and wherein one further data item holds said generated report; and providing said distinguishable quantity obtained by said processing during the processing step. When performing the suggested method, a data structure is made available, which identifies the processing and/or a property of the distinguishable quantity of the secondary product. The data structure can be transferred to a next processing phase together with the associated distinguishable quantity. Thus, the at least one property of the secondary product is known with increased precision.

According to an aspect of the invention, a method for processing at least one primary product into at least one secondary product is suggested. The method includes: providing at least one primary product; processing the at least one primary product into a distinguishable quantity of a secondary product by a processing device; generating a report by a state reporting device associated with the processing device, wherein said report includes at least one state value indicative of a processing state during the processing step; generating a unique object identifier indicative of said distinguishable quantity, which distinguishable quantity is obtained by said processing during the processing step; generating a data structure having at least two data items, wherein one data item holds the object identifier, and wherein one further data item holds said generated report; signing the data structure by setting a data item of the data structure such that it holds a signature indicative of natural or legal person and/or a group thereof; and setting a data item of the data structure such that it holds a cryptographic representation of the signed data structure; and providing said distinguishable quantity obtained by said processing during the processing step. When performing the suggested method, a data structure is made available, which identifies the processing and/or a property of the distinguishable quantity of the secondary product. The data structure can be transferred to a next processing phase together with the associated distinguishable quantity. Thus, the at least one property of the secondary product is known with increased precision. Further, the signature preferably is an electronic signature. The signature usually is a verifiable, such that it is verifiable which legal or natural person and/or which group has signed the data structure. There are many well-established methods for signing data, which are could advantageously be employed here. Signing data usually serves data security purposes.

The processing device may include an arrangement and/or a series of processing devices. A processing device may be any device that is configured to process the at least one primary product into at least one secondary product. Preferably, the primary product is processed into the secondary product by means of the processing device.

The primary product preferably differs from the secondary product in at least one product quality. A product quality may be a chemical, physical, and/or mechanical property of the product. A product quality of the secondary product is affectable by the recorded state of the processing by the processing device.

The data structure is preferably configured to represent a production process performed during producing the distinguishable quantity of the secondary product. The data structure is associated with one processing and/or one distinguishable quantity from said processing. This association is made by the unique object identifier. Thus, this object identifier is the own object identifier of the data structure, as it identifies and/or indicates the object of the data structure, which is specified in-depth by the remaining data items.

A state reporting device may be configured to monitor a state of the processing and to report the monitored state and/or a calculation result based on the monitored state. Examples for state reporting devices include a sensor, including a so-called edge device, a driver, an actuator, and/or an evaluation software connected in series therewith. The report may include a time series of state values, as this increases a precision of the report. If there are more than one process states reported, then it is preferred to have multiple synchronized time series of different or distinguishable state values. This step facilitates an optional step of deriving a state value of the processing. The at least one process state during the step of processing the at least one primary product into at least one secondary product is recorded. That is, the state values express the conditions, during which the secondary product was obtained. It is preferred, if the record is generated during the processing step. However, there may be applications of this method, in which is it preferrable to generate or at least finalize the record after the second product is obtained. For example, a backwards filter may be applicable only after a time series is completed.

Optionally, during said providing said distinguishable quantity may be separately provided, and the method may include: linking the separated distinguishable quantity to the generated data structure. As the processed distinguishable quantity is provided in a separated manner and is linked to the data structure, the knowledge of the property or properties and the distinguishable quantity in a distinguishable manner are both available individually and in combination. Providing the distinguishable quantity separately includes at least separating the distinguishable quantity. Separately providing may include providing in a transportable form.

Linking may be understood as creating a bidirectional connection of the separated quantity with the generated data structure. The quantity of the secondary product is a physical or “real world” entity. The data structure can be a physical or “real world” entity, such as a writing on a paper, or it can be a virtual entity, such as an entry in a computer-stored database, wherein the virtual data structure is preferred. A bidirectional connection may mean that one can find/access the data structure starting from the quantity and one can find/locate the quantity starting from the data structure.

Said separately providing may optionally include: packaging into a package said distinguishable quantity, that is obtained from said processing step, i.e. the distinguishable quantity of the secondary product. If the distinguishable quantity is packaged, it is easier to identify this quantity. “A package” preferably includes one package or at least one package.

Optionally, said linking includes attaching a link object to the package. Thus, a user can follow the attached link to the data structure. This aspect is for example beneficial in handling the distinguishable quantity. Preferred examples for attaching said link include: printing the link object on the package, writing the link object to a data storage attached to the package, and/or providing the link object within the package. Each of these preferred examples offers a unique combination of accessibility to the link and permanence of the link.

The method may optionally be applied to processes where during said processing step the at least one primary product is processed into multiple distinguishable quantities of the same secondary product and/or different secondary products, wherein at least the steps: generating a unique object identifier, generating a data structure, providing said distinguishable quantity, and, if applicable, the steps: linking the separated distinguishable quantity and attaching the link object, may optionally be performed for each of multiple of said distinguishable quantities. Thus, a precise knowledge of multiple distinguishable quantities of secondary product(s) can be gained.

Preferably, each quantity of a primary or secondary product is a distinguishable quantity.

According to one aspect of the invention, another method for processing at least one primary product into at least one secondary product is suggested. The method includes: providing at least one primary product; processing the at least one primary product into a distinguishable quantity of a secondary product by a processing device; generating a report by a state reporting device associated with the processing device, wherein said report includes at least one state value indicative of a processing state during the processing step; generating a unique object identifier indicative of said processing during the processing step; generating a data structure having at least two data items, wherein one data item holds the object identifier, and wherein one further data item holds said generated report; and providing said distinguishable quantity obtained by said processing during the processing step. When performing the suggested method, a data structure is made available, which identifies the processing and/or a property of the distinguishable quantity of the secondary product. Thus, at least one property of the secondary product, that results from said processing, is implicitly knows in the data structure, and it can be transferred together with the distinguishable quantity to a next processing phase.

According to another option, the above methods may be combined, such that the object identifier is indicative of the said processing during the processing step and of the distinguishable quantity of the secondary obtained from said processing during the processing step. This method may be advantageously applied for example in cases, where several secondary products are obtained from the same processing.

The following options apply to the above method where the object identifier is indicative of the processing as well as to the above method where the object identifier is indicative of the distinguishable quantity.

Now, some method options are laid down, which may be applied during or after said data structure generation. Especially in this regard, a “setting a data item” of the data structure such that it holds a specific value may include one of: generating the data item while generating the data structure and assigning the specific value to the data item, expanding the data structure by adding the data item and assigning the specific value to the data item, and/or providing the generated data structure having the data item and assigning the specific value to the data item.

According to a preferred option, said providing said obtained distinguishable quantity includes: providing the separated distinguishable quantity at an identifiable location; and wherein the method includes: setting a data item of the data structure such that it holds a location identifier indicative of the identifiable location. Thus, a user can easily locate a distinguishable quantity of the product having specific product properties.

The method also may include: providing a data owner indicator indicative of a natural or legal person and/or a group thereof; and setting a data item of the data structure such that it holds the data owner indicator. The data owner indicator preferably represents one or more persons responsible for holding, maintaining, updating, amending, and/or the like of the data structure. This feature makes it easier to have the precise knowledge centralized, like stored availably on a server, with clear responsibility. Thus, handling the data structure does not become more difficult when access to at least one of many data structures is granted to at least one of many parties.

The method may include: providing a distinguishable quantity owner indicator indicative of a natural or legal person and/or a group thereof; and setting a data item of the data structure such that it holds the distinguishable quantity owner indicator. This feature realizes the task to describe an economic property of the distinguishable quantity. However, opposed to a concept of having a parallel data structure for economic properties of the distinguishable quantity in addition to the above-described data structure for process properties, it is easier to handle and saves space and computation power to include all features in one data structure. That is, there is a technical advantage of saving resources in the technical solution of handling technical and economical properties in a common data structure.

Further, the method may include: setting a data item of the data structure such that it holds a processing device identifier indicative of the processing device used to obtain the distinguishable quantity. If there are two separate processing devices for the same task, then it is highly likely that a secondary product processed in the one processing device differs from a secondary product processed in the other processing device. However, it is also possible that the state recording device may not be able to perceive or record these differences and/or it is possible that it is not yet known at the time of processing which difference has which impact later on. Thus, there are many use cases where it is beneficial to know which processing device was involved in obtaining a distinguishable quantity.

The method may also include: setting a data item of the data structure such that it holds a processing time indicator indicative of when the distinguishable quantity was produced. Preferably, the processing time indicator includes a start time of the processing, such that another state value might in retrospect be traceable from device logs or the like. Preferably, the processing time indicator includes a stop time of the processing, such that an age of the product is determinable. Optionally, the method may include: determining a production timestamp indicative of a time of said processing step and/or said step of separately provided; and setting a data item of the data structure such that it holds said production timestamp.

Preferably, the method may include: setting a data item of the data structure such that it holds a state reporting device identifier indicative of the state reporting device used to obtain the report. This information, too, allows to take into account individual sensitivity differences between different state report devices.

There might be the case where a primary product may not be delivered in distinguishable quantity, for example via a buffer tank or by pipeline. For example, in these cases, it may be advantageous if said step of providing at least one primary product included: providing a lot of a primary product and a primary product lot identifier indicative of a lot of the primary product; and wherein the method further includes: setting a data item of the data structure such that it holds the primary product lot indicator.

There might be the case where a data structure is to be shared with a competitor, customer, and/or any other 3rd party, in which case a confidentiality cannot be guaranteed. If the method included: encrypting at least one data item, then confidentiality regarding the encrypted data item(s) content can be maintained.

In order to increase a knowledge regarding a specific distinguishable quantity, said method may include: generating another report by another state reporting device associated with the processing device, wherein said other report includes at least one further state value indicative of a processing state during the processing step; and setting a data item of the data structure such that it holds said other generated report.

In order to further process the secondary product, a classification or the like of the secondary product can be important. For example, it might be necessary to use only secondary products from a specific bin within a specification for the secondary product, or to mix secondary products of different bins to equally reflect a whole specified range, in order to obtain a desired quality of a next level product. Thus, the method may include: providing a statistical ruleset configured for determining a statistical measure of the at least one report; determining a single statistical measure based on the at least one report and the statistical ruleset; and setting a data item of the data structure such that it holds said single statistical measure.

Further, the method may include: signing the data structure by setting a data item of the data structure such that it holds a signature indicative of a natural or legal person and/or a group thereof; and setting a data item of the data structure such that it holds a cryptographic representation of the signed data structure. The signature preferably is an electronic signature. The signature usually is a verifiable, such that it is verifiable which legal or natural person and/or which group has signed the data structure. There are many well-established methods for signing data, which are could advantageously be employed here. Signing data usually serves data security purposes.

Further, the method may include: determining a cryptographic representation of the data structure associated with the distinguishable quantity of the primary product; and setting a data item of the secondary data structure such that it holds said cryptographic representation of the data structure associated with the distinguishable quantity of the primary product. This option enables a blockchain, which is beneficial in bigger ecosystems, where many parties contribute data structures.

Optionally, the method may include: providing at least one distinguishable quantity of a primary product; providing, for each provided distinguishable quantity of a primary product, a data structure associated with the distinguishable quantity, wherein said data structure includes one data item holding an object identifier indicative of the distinguishable quantity of the primary product, and one further item holding a report including at least one state value indicative of a processing state of a process performed during obtaining the primary product of the distinguishable quantity indicated by the object identifier; and generating the (new) data structure, which was mentioned in the above options, such that it also includes a further data item holds the object identifier indicative of the distinguishable quantity of the primary product. This option allows to additionally access not only the process state(s) of the latest processing, but also a history of process state(s) of pre-processing(s). All these process states are individual and specific for the secondary product of the distinguishable quantity, which is indicated by the own object identifier held in the newly generated data structure.

According to an aspect of the invention, a method for processing at least one primary product into at least one secondary product is suggested, which includes: providing at least one primary product; providing access to a database that is configured to include multiple data structures; processing the at least one primary product into a distinguishable quantity of a secondary product by a processing device; generating a report by a state reporting device associated with the processing device, wherein said report includes at least one state value indicative of a processing state during the processing step; generating a unique object identifier indicative of said distinguishable quantity, which distinguishable quantity is obtained by said processing during the processing step; generating a data structure having at least two data items, wherein one data item holds the object identifier, and wherein one further data item holds said generated report; adding or entering into the database the data structure associated with the distinguishable quantity of the secondary product; and providing said distinguishable quantity obtained by said processing during the processing step. A data set is useful for example not only for adjusting parameters in a directly succeeding processing step, but for it may be used in many following processing steps until a final product and even afterwards during recycling the final product and/or recycling of excess product. All these process steps may be performed by different entities at different times and locations, wherein often a succeeding steps might even be unknown at the time of producing a certain distinguishable quantity. By adding the data structure(s) into the database, the content(s) of the data structure(s) is/are reliably provisioned, such that the precise product property knowledge is reliably procured. Thus, it is easily and reliably retrievable by any following process step.

In the art are known many database techniques. Any database technique may be employed to realize this database, as long as it accessibly holds the data structures. Preferably, a distributed database, such as a cloud service, may be used. In order to easily communicate data structures with many participants, an internet-based database is preferred.

Although the focus of the data structure as well as of the database is on providing technical properties for technical processes, it may be essential to recognize that also a per-se non-technical information, such as a location or an ownership of a specific distinguishable quantity, can have a strong technical impact, for example in material handling and/or in acceleration adjustments of continuous processing devices. According to an aspect of the invention, a method for processing at least one primary product into at least one secondary product is suggested, including the steps of: providing at least one primary product; providing access to a database that is configured to include multiple data structures; processing the at least one primary product into a distinguishable quantity of a secondary product by a processing device; generating a report by a state reporting device associated with the processing device, wherein said report includes at least one state value indicative of a processing state during the processing step; generating a unique object identifier indicative of said distinguishable quantity, which distinguishable quantity is obtained by said processing during the processing step; generating a data structure having at least two data items, wherein one data item holds the object identifier, and wherein one further data item holds said generated report; generating a status indicator indicative of a current status of a variable factor of the distinguishable quantity indicated by the object identifier, wherein the current status is at least one selected status of a group consisting of: an expected production date, a storage location, an ownership, a reservation, an intended disposition, an intended usage, a percentage of remaining quantity, an actual disposition, an actual usage, and a price; setting a data item of the data structure associated with the secondary product such that it holds said status indicator; adding or entering into the database the data structure associated with the distinguishable quantity of the secondary product; and providing said distinguishable quantity obtained by said processing during the processing step. The status indicator may include the at least one current status.

Optionally, the method further includes: providing at least one distinguishable quantity of a primary product; providing, for at least one provided distinguishable quantity of a primary product, a data structure associated with the distinguishable quantity, wherein said data structure includes one data item holding an object identifier indicative of the distinguishable quantity of the primary product, and one further data item holding a report including at least one state value indicative of a processing state of a process performed during obtaining the primary product of the distinguishable quantity indicated by the object identifier; setting a data item of the secondary data structure such that it holds the object identifier indicative of the distinguishable quantity of the primary product; and adding into the database the data structure associated with the distinguishable quantity of the primary product. Thus, the data structure can reflect not only the state values of one processing but, but it can point and/or refer to a data structure that reflect a state value of a preceding process step. This option thus provides a deeper and more precise technical specification of a certain product. Preferably, the method further includes adding the primary data structure to the database.

According to an aspect of the invention, a method for processing at least one primary product into at least one secondary product is suggested. The method includes the steps: providing at least one distinguishable quantity of a primary product; providing, for at least one provided distinguishable quantity of a primary product, a data structure associated with the distinguishable quantity, wherein said data structure includes one data item holding an object identifier indicative of the distinguishable quantity of the primary product, and one further data item holding a report including at least one state value indicative of a processing state of a process performed during obtaining the primary product of the distinguishable quantity indicated by the object identifier; processing the at least one primary product into a distinguishable quantity of a secondary product by a processing device; generating an unique object identifier indicative of said distinguishable quantity of the secondary product, which is obtained by said processing during the processing step; generating a new data structure having at least two data items, wherein one data item holds the generated object identifier indicative of the distinguishable quantity of the secondary product, and wherein one further data item holds the object identifier indicative of the distinguishable quantity of the primary product; and providing said distinguishable quantity obtained by said processing during the processing step. This method, too, provides a deeper and more precise technical specification of a certain product: Performing the method of this aspect thus generates a chain of data structures, which are associated with each other by citing the object identifier(s) of the primary product(s). Thus, a data structure of a secondary product points towards a history or a log of process state values of process steps performed previous to this process step.

This method allows an understanding of properties of the secondary product, which result from the processing step from the primary product into the secondary product. This method also allows a deep understanding of properties of the secondary product, which do not result from the processing step from this primary product to this secondary product, but from knowing one or more precise properties of the primary product. After having performed the method, one has three entities/results, which are: the data structure associated with the distinguishable quantity of the primary product, the new distinguishable quantity of the secondary product, and the new data structure holding the generated object identifier indicative of the distinguishable quantity of the secondary product. That is, the method results in a chain of digital twins of products, where a digital twin of a secondary product references to a digital twin of a primary product. Having a chain of digital twins is advantageous as a property of the distinguishable quantity of the primary product may be known only after the generation of the secondary product, for example due to a duration of a property test or where a property is only known (detectable) from the secondary product, in which cases the chain of digital twins maintains a single and thus unambiguous data base.

A “distinguishable quantity” of a product is a general term for a specific or particular unit of this product. A “distinguishable quantity” has an amount, such as a weight, a volume, a particle number, and/or a mol number, which defines a size of this quantity. A “distinguishable quantity” also has a distinguishability, which is another property, such that a user could distinguish, at least at one point of the processing, this quantity from another quantity. This distinguishability may follow from the quantity being provided in a container and/or during a specific time. For example, a distinguishable quantity may be provided from a first processing device on/in a conveyor means between two defined points of time.

The provided data structure associated with the distinguishable quantity of the primary product may be referred to as a primary data structure or a first data structure, while the generated new data structure is associated with the distinguishable quantity of the secondary product and may thus be referred to as a secondary data structure or second data structure.

According to an option, the second data structure may be generated such that it has, besides the data item holding the object identifier indicative of the distinguishable quantity of the primary product, only other or different data items than the data items of the provided data structure associated with the distinguishable quantity of the primary product. This option means that the data structures forcedly have unambiguous data items, which is beneficial for data reliability.

According to an option, the method may include: providing access to a database, that is configured to include multiple data structures each having at least two data items and is configured to provide access to a data structure such that any data item of the accessed data structure is settable; and adding into the database the data structure associated with the distinguishable quantity of the secondary product. The feature of the data item being “settable” includes that the data item is addable to the data structure, is amendable within the data structure and is removeable from the data structure. In other words, the method may include: providing access to a data-base, that is configured to include multiple data structures each having at least two data items and is configured to provide access to a data structure such that a data item is addable the accessed data structure, is amendable within the accessed data structure and is removeable from the accessed data structure; and adding into the database the data structure associated with the distinguishable quantity of the secondary product.

One or more added and/or amended data items may hold: a current location of the distinguishable quantity.

One or more added and/or amended data items may hold: a current owner and/or an owner history of the data structure, a current owner and/or an owner history of the data item, and/or a current owner and/or an owner history of the distinguishable quantity indicated by the data structure's own object identifier.

One or more added and/or amended data items may hold: a timestamp (see below), a transportation duration, a process value of a transportation process (e.g. a temperature during transportation), past and/or present position data of the distinguishable quantity (e.g. a tracking of a transportation).

One or more added and/or amended data items may hold: process data of a testing process for testing a sample of the distinguishable quantity.

One or more added and/or amended data items may hold: a role information indicative of a permission for a natural and/or legal person and/or a group thereof, which role information is also indicative of a permission level regarding this data structure and/or one or more defined data items of this data structure. For example, different persons may be allowed to read different data items. For example, a data owner may allow a test device operator to amend a certain data item, which is reserved for test information.

One or more added and/or amended data items may hold: a provision information regarding the distinguishable quantity indicated by the own object identifier, such as an encryption provision for the data structure and/or for data items thereof, a handling provision, a storage provision, and/or a criterion for exclusion.

One or more added and/or amended data items may include a certificate information and/or a certificate expiry information, which preferably are signed, preferably cryptographically signed, data items. A “fairtrade” certificate is an example for such a certificate information.

One or more added and/or amended data items may hold: commercial information and or a history thereof regarding the distinguishable quantity indicated by the own object identifier, such as a manufacturing cost, a production manhours information, a sales price, an offered sales price, an actually achieved sales price, and/or an exchange rate at a sales date.

One or more added and/or amended data items may hold: manufacturing tool data indicative of one or more manufacturing tool used during manufacturing/processing the distinguishable quantity indicated by the own object identifier.

One or more of the above data items may also be originally added to the data structure when the data structure is originally generated.

Thus, the generated data structure is held in a database such that additional data items can subsequently be added to the data structure. This is useful, for example, where a test result of a sample of the distinguished quantity is available only after the production of the distinguished quantity.

The generated data structure is held in a database such that a data item can be amended. This is useful, for example, where a status or property of the distinguishable quantity is variable, such as a location or a concentration of a volatile component.

The generated data structure is held in a database such that a data item can be deleted from the data structure. This is useful, for example, where a tatus or property of the distinguishable quantity is obsolete, like a storage location when the complete distinguishable quantity has been used to produce a secondary product.

Adding, amending and/or removing only a selected data item implies that the remainder of the data structure remains. This invention targets a use case where the data structure is used during succeeding steps in the life cycle of the distinguishable quantity of the product. Thus, to only set (i.e. add, amend, and/or remove) single data items it is advantageous over a blockchain or the like, as it saves storage space. In addition, there is no “more up to date” version of the data structure, which can save time when searching the data structure.

According to an option, the method may additionally or alternatively include: providing access to a database, that is configured to include multiple data structures; accessing via the database one data structure held by the database; and setting a data item of the accessed data structure. In other words, the method may include: providing access to a database, that is configured to include multiple data structures; accessing via the database one data structure held by the data-base; and adding a data item to the accessed data structure, amending a data item of the accessed data structure and/or removing a data item from the accessed data structure.

According to an option, the method may include: providing access to the database; accessing via the database one data structure held by the database; and reading a data item from the accessed data structure. This may be used to adjust a process parameter of a secondary production process, for example. This may also be used to select a distinguishable quantity from multiple distinguishable quantities for a secondary processing based on the specific properties of the product, which are reflected in the process states indicated by the read data item.

According to one option, the method may include: providing a pre-set characteristics ruleset, which specifies at least one characteristic value determination rule configured to determine at least one characteristic value based on at least one record, and which specifies at least one record applicable to the characteristics ruleset; and determining at least one characteristic value by applying the at least one record specified by the characteristics ruleset to at least one rule specified by the characteristics ruleset. That option is useful for a customer to decide whether a distinguishable quantity fits a pre-set technical requirement without disclosing all process data. Preferably, the method further includes: setting a data item of the data structure such that it holds the at least one determined characteristic value. Thus, the determination can be stored and retrieved without the need to spend again computation time for repeating a task.

In order to improve a reliability and/or acceptance of the report(s) included in the data structure, said method may include: taking a sample from the distinguishable quantity of the secondary product; generating a test result indicative of at least one material property of the secondary product of the processed and/or separated distinguishable quantity; generating a test result indicator indicative of the generated test result; and setting a data item of the data structure such that it holds the test result indicator. Preferably, the sample is taken in a time frame from a beginning of the processing until after the distinguishable quantity is separately provided.

The sample may be taken during and/or immediately after the step of processing the distinguishable quantity of the primary product to the distinguishable quantity of the secondary product. Then, there is the case where the generation of the test result indicator happens so fast that the data structure can be added to the data base originally including the data item holding the test result indicator. There is also the case where the generation of the test result indication takes some time such that the respective data structure has been added to the data base and the method has: accessing the data structure via the data base; and adding/amending the data item holding the test result indicator.

Optionally, the method may have: taking this sample based on a schedule, accessing the data structure via the data base; and adding/amending the data item holding the test result indicator. Thus, a product quality can be monitored.

Optionally, the method may have: taking this sample in response to a user request, accessing the data structure via the data base; and adding/amending the data item holding the test result indicator. Thus, a product quality information can be updated upon a request. For example, a potential buyer may request a detailed information (primary production process state data) on one or multiple distinguishable quantities of a primary product to control a secondary production process or a quality of a secondary product.

Another benefit is the case where a quality of the distinguishable quantity of the primary product is known only subsequently from secondary production process parameters. For example, a secondary process temperature may serve as a basis to conclude a chemical composition of the distinguishable quantity of the primary product. A use case may be to evaluate a course of the chemical compositions of multiple distinguishable quantities of the primary product in order to use this course as a basis for adjusting a process parameter of the primary production process in the future. This relates to chemical product parameters, physical product parameters, chemical process parameters, and physical process parameters as well.

In an alternative view, on may regard: a pre-tested sample as a primary product, a tested sample as a secondary product, a testing of a sample from the distinguishable quantity as processing the pre-tested sample into the tested sample by the testing equipment, and a generating of a test report by test report generator as generating a report by a state reporting device. For example, the testing equipment may be a container and the test report generator may be a temperature probe in the container. Accordingly, the testing of a sample from the distinguishable quantity and storing the test result to the data structure associated thereto may be accomplished by a method, which includes these steps: providing access to a database, that is configured to include multiple data structures; providing at least one primary product, which is a pre-tested sample/sample to be tested; providing via the database access to a data structure having at least a data item, that holds an own object identifier indicative of a distinguishable quantity of the primary product from which the sample is taken; processing the at least one primary product (sample) into a distinguishable quantity of a secondary product by a processing device, wherein the distinguishing quantity of the secondary product is a tested sample and the processing device is a testing equipment; generating a report by a state reporting device associated with the processing device, wherein said report includes at least one state value indicative of a processing state during the processing step, wherein the report is a test report, wherein the state reporting device is a test report generator, and wherein the state value is a test result; generating a new unique object identifier indicative of said distinguishable quantity, which distinguishable quantity is obtained by said processing during the processing step; generating a new data structure having at least two data items, wherein one data item holds the generated new object identifier, and wherein one further data item holds said generated report; adding the generated new data structure to the database; and setting another data item of the data structure, which has the data item, that holds the own object identifier indicative of the distinguishable quantity of the primary product from which the sample is taken, such that this other data item holds the generated new object identifier. Then, the test result can be stored separately but linked to the data structure of the sampled quantity. A use case may be when many parties need access to the data structure of the sampled quantity, but it is not desired that every party can read the test result.

According to a related aspect of the invention, a method for generating a data structure is suggested. Said method includes providing access to a database that is configured to include multiple data structures; providing at least one report that includes at least one state value indicative of a processing state during a processing step; providing a status indicator, indicative of a current status of a distinguishable quantity obtained by a process, of which a processing state is indicated by the at least one report; generating an unique object identifier indicative of the distinguishable quantity of a product, that is obtained by performing the processing of which the report indicates a processing state; generating a data structure having at least two data items, of which: one data item holds the generated object identifier, at least one further data item holds the provided report; and adding or entering into the database the data structure associated with the distinguishable quantity of the primary product. By entering the data structure(s) into the database, the content(s) of the data structure(s) is/are reliably provisioned, such that the precise product property knowledge is reliably procured. Thus, the precise product property is easily and reliably retrievable and usable by any following process step.

Each method described herein may be applied in many technical fields. However, preferably, each method described herein is applied to the field of chemistry. Thus, preferably, the primary product is a chemical product and/or a chemical raw material, and the secondary product is a chemical product. A chemical product is defined by a chemical description and optionally a physical, electrical and/or mechanical description.

According to another aspect of the invention, a method for processing at least one primary product into a secondary product is suggested. The suggested method includes: providing at least one distinguishable quantity of a primary product; providing, for each provided distinguishable quantity of a primary product, a data structure associated with the distinguishable quantity, wherein said data structure includes one data item holding an object identifier indicative of the distinguishable quantity of the primary product, and one further item holding a report including at least one state value indicative of a processing state of a process performed during obtaining the primary product of the distinguishable quantity indicated by the object identifier; processing the at least one primary product into a distinguishable quantity of a secondary product by a processing device; generating a unique object indicative of the distinguishable quantity of the secondary product; generating a new data structure having at least two data items, wherein one data item holds the generated object identifier indicative of the distinguishable quantity of the secondary product, and wherein one further data item holds the object identifier indicative of the distinguishable quantity of the primary product; and providing said distinguishable quantity obtained by said processing during the processing step. Performing the method of this aspect thus generates a chain of data structures, which are associated with each other by citing the object identifier(s) of the primary product(s). Thus, a data structure of a secondary product points towards a history or a log of process state values of process steps performed previous to this process step. This method thus allows a deep understanding of properties of the secondary product, which do not result from the processing step from this primary product to this secondary product.

According to an option, the method of this aspect may include: generating a report by a respective state reporting device associated with the processing device, wherein said report includes at least one state value indicative of a processing state during the processing step; and setting a data item of the secondary data structure, such that it holds said generated report. Thus, the data structure informs about one or more process states of this processing step as well as it points to one or more pre-process states via the primary product object identifier.

Also in this method, it is optionally possible to provide a lot of a primary product. In this case it is preferred to include in the method: providing a primary product lot identifier indicative of said lot of the primary product; and setting a data item of the data structure such that it holds the primary product lot indicator. Thus, it is possible to include detailed information also on one or more primary products, on which no data structure is supplied. Again, technical properties of the secondary product are more precisely rendered, which paves the way for multiple technical improvements.

Preferably, a data item of the primary data structure holds a production timestamp indicative of a time when said primary product was produced. In order to provide a user with an age of the primary product as a product property, said method may include: determining an age indicator indicative of a duration from the time indicated by said production timestamp until a time when the primary product is provided; and setting a data item of the data structure associated with the primary product such that it holds said age indicator, and/or setting a data item of the data structure associated with the distinguishable quantity of the secondary product such that it holds said age indicator linked with the object identifier indicative of the distinguishable quantity of the primary product.

There may be a case, where different distinguishable quantities of the same primary product and the same number of primary data structures are available, wherein each of these primary data structures is associated with a different one of these distinguishable quantities of the primary product, wherein each primary data structure has a data item holding a production timestamp indicative of a time when the respective primary product was produced. Preferably, the method includes performing, in this case, the following steps: determining for each distinguishable quantity of the primary product an age from the time indicated by said respective production timestamp until a present time; and choosing at least one of said distinguishable quantities of the primary product based on its respective age and a pre-set ruleset; wherein the chosen distinguishable quantity of the primary product or distinguishable quantities of the primary product is/are provided in said providing step. Thus, a primary product can be selected based on its respective age. Thus, an old product can be selected before it degrades too much and becomes out-of-specification. It is possible to wait until an undesired volatile component of the primary product is reduced in concentration. A distinguishable quantity of a primary product may also be referred to as a primary (distinguishable) quantity. A distinguishable quantity of a secondary product may also be referred to as a secondary (distinguishable) quantity.

In order to increase a recycling performance, wherein said secondary product is a final product; the method may include: attaching an identifier device to said secondary product, wherein said identifier device is indicative of the object identifier of the secondary data structure. Thus, a recycling process may include to read the identifier device, such as an RFID tag, a serial number, and/or a QR code at the final product and/or inside of the final product, and then determine an exact composition and/or property of the final product, such that a quality of a recycling process product is improved.

The aspects and options within this invention description can be combined. Thus, the method of this option may additionally include the method steps of any other method or option mentioned above and/or below to achieve the same advantageous effect.

Another aspect of the invention refers to a data structure having at least two data items, wherein: one data item holds an unique object identifier, that is indicative of a processing during a processing step and/or of a distinguishable quantity obtained by said processing during said processing step; and one further data item holds a report, which includes at least one state value indicative of a processing state of said processing step. This data structure is a means for more precise knowledge of a secondary product, as it is holds the report and the object identifier in mutual association.

The data structure may include a data item holding a package identifier indicative of the at least one package having said distinguishable quantity indicated by the object identifier. Thus, a link between the data structure and the distinguishable quantity can easily be included into the data structure.

The data structure may include a data item holding a location identifier indicative of an identifiable location of said distinguishable quantity indicated by the object identifier. Thus, the distinguishable quantity can easily be found and handled.

The data structure may include a data item holding a data owner indicator indicative of a natural or legal person and/or a group thereof owning this data structure. Thus, even in complex scenarios with many data structures from many sources and with many data structure consumers, it is easily documented who is responsible for maintaining said data structure.

The data structure may include a data item holding a distinguishable quantity owner indicator indicative of a natural or legal person and/or a group thereof owning said distinguishable quantity indicated by the object identifier. Thus, an ownership of the distinguishable quantity is easily documented.

The data structure may include a data item holding a primary product lot identifier indicative of a lot of a primary product to the secondary product of said distinguishable quantity, that is indicated by the object identifier. Thus, a user can easily trace which lot of a primary product has been used for which distinguishable quantity of the secondary product. This option allows to fulfill legal regulations concerning source documentation, even in said complex scenarios.

The data structure may include a data item holding a test result indicator indicative of the generated test result based on a sample taken from said distinguishable quantity indicated by the object identifier. This renders more precisely the product properties of the secondary product.

The data structure may include a data item holding another report, that is configured to include at least one state value indicative of a processing state of a step of processing at least one raw material into said distinguishable quantity of said product. This again increases product property knowledge.

The data structure may include a data item holding a characteristic value and/or a series of characteristic values, that is characteristic for the at least one report. That option is useful for situation where a customer wants to decide whether a distinguishable quantity fits a pre-set technical requirement but a procurer wants to not disclose some or all process data.

The data structure may include an encrypted data item. This can guarantee confidentiality.

The data structure may include a data item may include a signature configured to sign the data structure or at least a part thereof; and a data item may hold a cryptographic representation of the signed data structure. This can guarantee authenticity of the precise property knowledge contained in the data structure.

The data structure may include a data item that holds a cryptographic representation of a data structure associated with a distinguishable quantity of a primary product, that is a predecessor to the distinguishable quantity of the product indicated by the own object identifier. This option allows a blockchain feature such that it is possible for a customer to reliably reproduce a history of predecessor products and their properties. One use case might be a carbon footprint determination.

The data structure may have at least two data items, wherein: one data item holds a second object identifier, that is configured to be a unique object identifier for identifying a processed distinguishable quantity of a product; at least one data item holds a first object identifier, that is configured to be a unique object identifier for identifying a distinguishable quantity of a primary product processed to a distinguishable quantity identified by the second object identifier. Thus, the data structure technically represents technical features of the secondary product, which may originate from the technical process of obtaining said secondary product, as well as one or more pre-processes thereof. In the result, a more precise technical description can be achieved.

According to an aspect of this invention, a data structure may have at least two data items, wherein: one data item holds a second object identifier, that is configured to be a unique object identifier for identifying a processed distinguishable quantity of a product; at least one data item holds a first object identifier, that is configured to be a unique object identifier for identifying a distinguishable quantity of a primary product processed to a distinguishable quantity identified by the second object identifier. Thus, the data structure technically represents technical features of the secondary product, which may originate from the technical process of obtaining said secondary product, as well as one or more pre-processes thereof. In the result, a more precise technical description can be achieved. The data structure of this aspect may have any features and advantages of any other data structure disclosed herein.

Another aspect of this invention is a data carrier signal carrying a data structure as described above, and/or as provided by the above-described method. This is beneficial for distributed systems, such as internet-connected manufacturing systems.

According to another aspect of this invention a database is suggested. The database includes or at least it is configured to include multiple data structures. Each data structure includes at least two data items, of which: one data item holds an own object identifier indicative of a distinguishable quantity of a product; and one further data item holds a report including at least one state value, and preferably a time series of state values, indicative of a processing state of a process, which process was previously performed during obtaining the product of the distinguishable quantity indicated by the own object identifier. By adding the data structure(s) into the database, the content(s) of the data structure(s) is/are reliably provisioned, such that the precise product property knowledge is reliably procured. Thus, the precise product property is easily and reliably retrievable and usable by any following process step. The database may preferably be computer-implemented.

Next will be presented methods using the data structure and/or the database in a next producing step. Thus, the above secondary product may now be referred to as a primary product for a following processing.

According to an aspect of the invention, another method for producing a secondary product from a distinguishable quantity of a primary product is suggested. The method includes: providing the above-defined data structure and/or the data structure as generated by any above method; providing the distinguishable quantity of a primary product, which distinguishable quantity is indicated by the own object identifier held by the data structure; reading from the data structure a report, which includes at least one state value indicative of a processing state of the primary product of the provided distinguishable quantity; adjusting a process parameter of a processing device based on the read report; and processing the primary product of the provided distinguishable quantity into the secondary product by means of the processing device or by the processing device. In other words: the method according to this aspect makes use of the documented precise technical properties of the product, which is associated with the data structure. By applying this method, it is possible to technically adapt a process to one or more technical properties of the primary product, thus generating a secondary product with advanced properties. The secondary product may have more evenly distributed properties, and/or it can be tuned to a tighter specification.

Another aspect of this invention is: using at least one data item of the above-defined data structure and/or of the data structure as generated by any above method for adjusting, especially as a basis for said adjusting, of a process parameter of a processing device for processing a distinguishable quantity of a primary product, which distinguishable quantity is indicated by the own object identifier held by the data structure. This use of the data structure and the associated distinguishable quantity has the same advantages as the above method.

Another aspect of this invention relates to a method for producing a secondary product from a quantity of a primary product. The suggested method includes: providing as a first data structure the above-defined data structure and/or the data structure as generated by any above method; providing the distinguishable quantity of the primary product identified by the object identifier held by a data item of the data structure; reading from the data structure a report, which includes at least one state value indicative of a processing state of the primary product of the provided distinguishable quantity; processing the primary product of the provided distinguishable quantity into the secondary product by means of a processing device or by a processing device; recording at least one of: a state value of the processing device during said processing step, a deviation value of the recorded state value from a target state value, a product property value of the secondary product during and/or after said processing step, and/or a deviation value of the recorded product property value from a target property value; and providing a data set including the at least on recorded value and the object identifier indicative of the provided distinguishable quantity of the primary product. The method according to this aspect has the advantageous effect to generate a data structure that can be used to improve the original or previous processing, where a primary product has been processed to a secondary product, based on an analysis generated during a later or follow-up processing, where the secondary product is processed into a tertiary product (a secondary product to the original secondary product). Thus, during a few process cycles, the original process can be optimized to the following process. In the end, an overall quality of the final product is improved. Preferably, the providing of the data set, which includes the recorded value, is part of a step of: generating a new data structure, such that the new data structure includes an unique object identifier indicative of the quantity of the secondary product and said data set. Preferably, the (remaining) steps of any above method are applied to this method, too, such that the generated second data structure also includes a unique object identifier indicative of the processed quantity of the second product.

One might understand this as a method for producing a second generation secondary product from a second generation primary product, wherein the second generation primary product is a first generation secondary product, wherein the data structure is a data structure according to any above method for processing at least one first generation primary product into the least one first generation secondary product, wherein the provided quantity is a quantity of the second generation primary product (i.e. the first generation secondary product), and wherein the second generation primary product (i.e. the first generation secondary product) is processed into the second generation secondary product. In other words, the terms “primary” product and “secondary” product preferably relate to one processing, and if there is a series of processing, then a secondary product of an earlier processing is a primary product of a succeeding processing.

According to a further aspect of the invention, a method for determining a characteristic value of a distinguishable quantity of a product is suggested. The method includes: providing a pre-set characteristics ruleset, which specifies at least one characteristic value determination rule configured to determine at least on characteristic value based on at least one record, and which specifies at least one record applicable to the characteristics ruleset; providing access to a data-base, that it is configured to include multiple data structures, wherein each data structure includes at least two data items, of which: one data item holds an own object identifier indicative of a distinguishable quantity of a product; and one further data item holds a report including at least one state value, and preferably a time series of state values, indicative of a processing state of a process, which process was previously performed during obtaining the product of the distinguishable quantity indicated by the own object identifier ; accessing via the database one data structure held by the database; reading at least one report held in an data item of the accessed data structure specified by the characteristics ruleset; determining at least one characteristic value by applying the at least one read record specified by the characteristics ruleset to at least one rule specified by the characteristics ruleset; and setting a data item of the accessed data structure such that it holds the at least one determined characteristic value. Preferably, said outputting includes setting a data item of the accessed data structure such that it holds the determined single degree of uniformity. The method of this aspect has the advantage of providing a single criterion for choosing a distinguishable quantity of a product which matches even very narrow specifications. Since this method has: setting a data item of the accessed data structure such that it holds the at least one determined characteristic value, the determined characteristic value can be stored and retrieved in order to save computation time in the future.

According to a further aspect of the invention, a method for determining a characteristic value of a distinguishable quantity of a product is suggested. The method includes: providing a pre-set characteristics ruleset, which specifies at least one characteristic value determination rule configured to determine at least on characteristic value based on at least one record, and which specifies at least one record applicable to the characteristics ruleset, wherein the pre-set characteristics ruleseMt includes at least two value ranges for each specified record, which ranges differ in at least one boundary value; and wherein the determined characteristic value is indicative of a bin defined by one of the value ranges; providing access to a database, that it is configured to include multiple data structures, wherein each data structure includes at least two data items, of which: one data item holds an own object identifier indicative of a distinguishable quantity of a product; and one further data item holds a report including at least one state value, and preferably a time series of state values, indicative of a processing state of a process, which process was previously performed during obtaining the product of the distinguishable quantity indicated by the own object identifier ; accessing via the database one data structure held by the database; reading at least one report held in an data item of the accessed data structure specified by the characteristics ruleset; determining at least one characteristic value by applying the at least one read record specified by the characteristics ruleset to at least one rule specified by the characteristics ruleset. Since this aspect has the at least two value ranges and the determined characteristic value is indicative of a bin defined by one of the value ranges, this option enables a binning of distinguishable quantities. In other words, each of these value ranges defines one bin for the product. A bin may be understood as a quality class within a quality classification.

This method may optionally include: performing in a case, where a data item of the data structure holds a object identifier indicative of a distinguishable quantity of a primary product, that is primary to the secondary product of the distinguishable quantity indicated by the own object identifier, the following steps, preferably in a recursive manner: accessing via the database a data structure, of which a data item holds an own object identifier that matches the object identifier of the primary product; and reading at least one report held in an data item of the accessed data structure specified by the characteristics ruleset; and wherein said at least one characteristic value is determines also by applying the at least one read record to at least one rule specified by the characteristics ruleset. These steps include previous process states into said determination of the single criterion, thus increasing a precision of choosing the best match between different distinguishable quantities of a product and a demand of a following process.

Preferably, the method may have: adding to a selection list an own object identifier held in a data item of the accessed data structure if a pre-set criterion is matched by the determined at least one characteristic value. Thus, if multiple data structures match the criterion, the results can be stored in a list in order to not again spending computation energy on this task, but rather investigating further only the results within the selection list.

A stricter approach with a probably smaller selection list result is achieved, when the own object identifier of the accessed data structure is added to the selection list only if a pre-set criterion is matched by each characteristic value determined from data items of this same data structure. One may understand this feature as a combination of selections. For example, a data structure may only be added to a selection list, if a data item A fits a criterion A′ and at the same time a data item B fits a criterion B.

According to another aspect of this invention, a method for producing a secondary product with a processing device is suggested, wherein the method includes: setting a characteristics ruleset for selecting a primary product to be processed; selecting at least one distinguishable quantity of the primary product by performing any above-described method for determining a characteristic value of a distinguishable quantity of a product based on the set requirements list; processing the primary product of at least one distinguishable quantity of the selected distinguishable quantity or selected distinguishable quantities into at least one distinguishable quantity of the secondary product. Here, only selected distinguishable quantities of primary product(s) are allowed for further processing, which allows a very fine control of a quality of the secondary product.

One main application of this invention is to find a quality of a product. This task is solved by a method for determining a product quality of a distinguishable quantity of a product according to another aspect of this invention. This suggested method includes: providing an object identifier indicative of a pre-selected distinguishable quantity; providing access to the above-described database; accessing a data structure, which includes a data item holding an own object identifier corresponding to the provided object identifier; determining at least one product quality of the primary product of the distinguishable quantity based on at least on data item included in the accessed data structure and a provided pre-set quality determination ruleset; and outputting the determines product quality. A quality may be any product property of combination of product properties derivable from the data structure. This method allows an evaluation derived from the processing state values. In other words, another layer of precise product properties can be accessed.

It is important to not forget that it is not always one complete distinguishable quantity of a primary product is processed into one complete distinguishable quantity of a secondary product. Thus, this method preferably takes processing quotas into account, which may be commonly known and/or set into a respective data item and be indicative of a quota of a specific distinguishable quantity of the primary product used to obtain the distinguishable quantity of the secondary product indicated by the own object identifier.

Preferably, said outputting includes outputting the determined product quality associated to the provided object identifier. For example, the quality and the object identifier are handed over to another method, and/or the method includes setting a data item of the data structure indicated by said object identifier such that this data item holds the determined product quality. Thus, the determination must not be repeated.

Optionally, this method may include: performing, preferably in a recursive manner: reading from the accessed data structure an object identifier indicative of a distinguishable quantity of a primary product to the product of the distinguishable quantity indicated by the own object identifier; accessing a data structure, which includes a data item holding the own object identifier indicative of the provided distinguishable quantity; determining at least one product quality of the primary product of the distinguishable quantity based on at least on data item included in the accessed data structure and a provided pre-set quality determination ruleset. An illustrative example for a product quality is a carbon dioxide footprint of a chemical product. If every preceding processing documents in the respective data structure the amount of exhausted carbon dioxide, then the total exhausted carbon dioxide for the distinguishable quantity can thus be determined.

Another aspect of this invention is that a method for producing a secondary product with a processing device is suggested, which includes: providing a distinguishable quantity of a primary product, which distinguishable quantity is linked to an object identifier indicative of the distinguishable quantity; performing the above method for determining a product quality, based on the object identifier; setting at least one process parameter of a processing device based on the at least one determined product quality; and processing at least a part of the primary product of the provided distinguishable quantity into at least a part of a distinguishable quantity of a secondary product by means of said processing device or by said processing device. This method uses the above determination method for adapting a process parameter to match the determined product quality. Thus, based on the precise product properties, an advanced secondary product may be obtained.

Another aspect of this invention refers to a computer program or a computer program product including instructions, wherein the instructions, when interpreted or executed by at least one processor unit, cause the at least one processor unit to perform any one of the above methods.

According to a further aspect, a computer-readable medium storing the above computer program instructions is suggested. The computer-readable medium is, in particular, a non-transitory computer-readable medium.

In embodiments, a computer-program or computer-program product comprises a program code for executing the above-described methods and functions by a computerized control device when run on at least one computerized device. A computer program product, such as a computer program means, may be embodied as a memory card, USB stick, CD-ROM, DVD or as a file which may be downloaded from a server in a network. For example, such a file may be provided by transferring the file including the computer program product from a wireless communication network.

Described in fewer words: A suggested method for processing at least one primary product into a secondary product includes the steps: providing at least one primary product; processing the at least one primary product into a distinguishable quantity of a secondary product by a processing device; generating a report by a state reporting device associated with the processing device, wherein said report includes at least one state value indicative of a processing state during the processing step; generating an unique object identifier indicative of said distinguishable quantity obtained by said processing during the processing step; generating a data structure having at least two data items, wherein one data item holds the object identifier, and wherein one further data item holds said generated report; and providing said distinguishable quantity obtained by said processing during the processing step. Another suggested method for processing a primary product into a secondary product includes the steps: providing at least one distinguishable quantity of a primary product; providing at least one a data structure associated with the distinguishable quantity of a primary product, said data structure including one data item holding an object identifier indicative of the distinguishable quantity of the primary product; processing the at least one primary product into a distinguishable quantity of a secondary product by a processing device; generating an unique object identifier indicative of said obtained distinguishable quantity; generating a new data structure having at least one data item holding the generated object identifier and one data item holding the object identifier indicative of the distinguishable quantity of the primary product; and providing said distinguishable quantity obtained during the processing step. Another suggested method for processing a primary product into a secondary product includes the steps: providing a primary product; providing access to a database configured to include multiple data structures; processing the primary product into a distinguishable quantity of a secondary product by a processing device; generating a report by a state reporting device associated with the processing device, said report being indicative of a processing state during the processing step; generating an unique object identifier indicative of said distinguishable quantity obtained during said processing step; generating a data structure having one data item holding the object identifier and one data item holding said report; adding into the database the data structure associated with the distinguishable quantity of the secondary product; and providing said distinguishable quantity.

Further possible implementations or alternative solutions of the invention also encompass combinations—that are not explicitly mentioned herein—of features described above or below in regard to the embodiments. The person skilled in the art may also add individual or isolated aspects and features to the most basic form of the invention.

Further embodiments, features and advantages of the present invention will become apparent from the subsequent description and dependent claims, taken in conjunction with the accompanying drawings, in which:

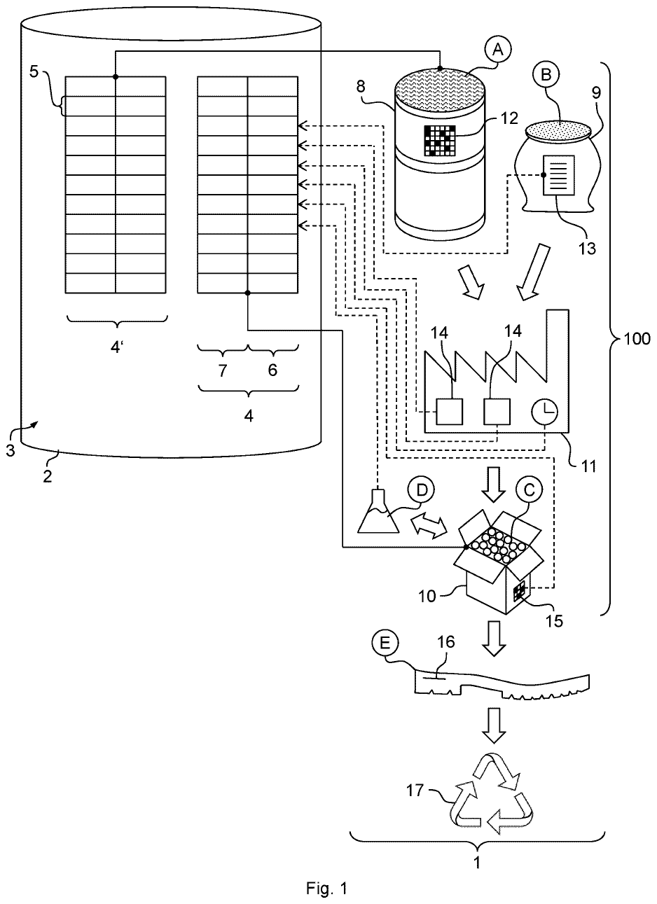

FIG. 1 schematically shows a scenario where a method according to an embodiment of the invention is implemented, and

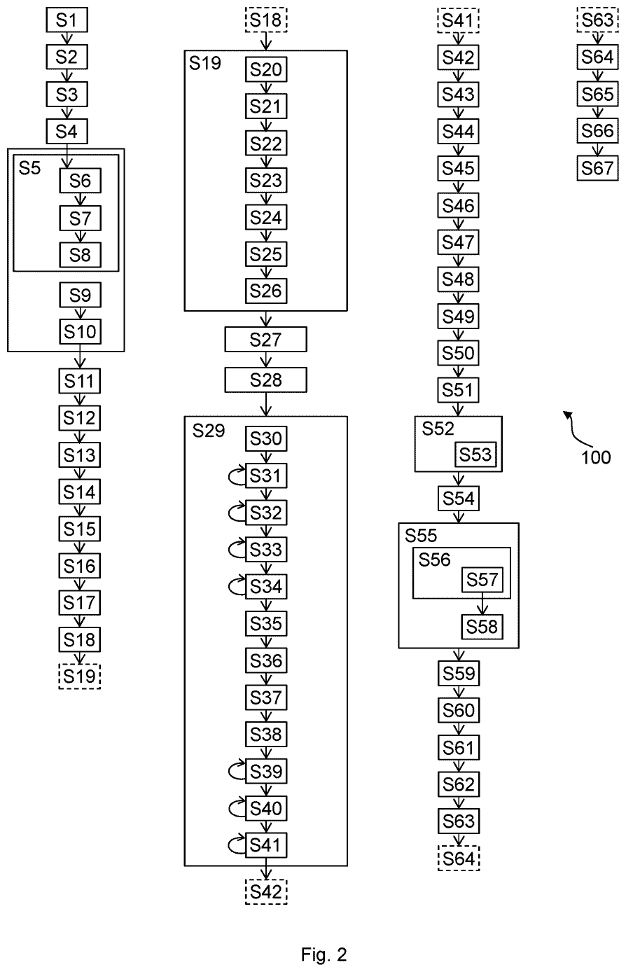

FIG. 2 shows a flow diagram of the method according to an embodiment of the invention.

In the Figures, like reference numerals designate like or functionally equivalent elements, unless otherwise indicated.

A use case for the invention is a marketplace for data structures. Each data structure reflects a processing performed during a life cycle of a product, wherein the life cycle includes primary products as well as recycling information. Each real life object, called a “distinguishable quantity” herein, is assigned with a unique object identifier. The object identifier links the real life object to the data structure.

The data structures hold processing parameters recorded during producing the respective object from primary products. Thus, the data structures do not or not only describe an object by a measured quality of the object itself but rather by means of a measured quality of a production process, which resulted in this object. Especially in the chemical industry, it is expected to gain much deeper and more precise information on a certain object from production process data than from object data itself.

The data structures are not or not completely encrypted. Rather, a depositary handles the data structures. The depositary protects the data structures and administrates access and manipulation of these data structures. The data structures thus do not form a blockchain where many parties can read each blockchain entry. One may think of the data structures handled by the depositary as investments or an account history handled by a bank. Since there is a depositary, there is no distributed ledger accessible to many parties (also the depositary may internally use such a technique for ensuring data integrity).

Next will be described an exemplary embodiment of the invention showing optional features for illustration purposes. FIG. 1 shows a scenario where the invention is applied. On the right side of FIG. 1 is depicted a product life cycle 1, and on the left is depicted a server 2 holding a database 3.

In this symbolic product life cycle 1, there are provided a liquid A in a barrel 8 and a powder B in a sack 9. These products A and B are industrially processed into a granulate C, which is packed in a box 10. A sample D is taken from the granulate C. The granulate C is later formed into a final product, such as a shoe sole E. After its product lifetime, the shoe sole E is disposed and recycled. The liquid A and the powder B are primary products to the granulate C. The granulate C is a secondary product to the liquid A and the powder B, and it is a primary product to the shoe sole E. That is, the terms “primary product” and “secondary product” are relative terms indicating a timely order and/or a predecessor-successor-order.

Next will be explained a method 100 for producing the granulate C from the liquid A and the powder B. However, this explanation serves illustration purposes mainly, and thus there are many optional steps included. Further, the shown sequence of steps is intended merely as a preferred example, but not as a necessary restriction.

In a first step S1, access to the database 3 is provided. The database 3 is configured to include multiple data structures 4.

A data structure 4 in this example is associated with a distinguishable quantity of a product. Each data structure 4 has at least one data item 5. Preferably, each data item 5 has a content 6 and a label 7 indicative of a type of the content. Each data structure 4 has one own object identifier. The object identifier is unique, and thus it is configured to indicate a distinguishable quantity of a product. The object identifier is held in one data item 5. It is possible that a data structure 4 holds one or more further object identifiers in other data items, for example as a reference to a primary product; however these one or more additional object identifiers are not the “own” object identifier. Due to the single and unique own object identifier one distinguishable quantity of a product is fixed as the associated distinguishable quantity.

The products of this example are the liquid A, the powder B, the granulate C, the sample D, and the shoe sole E. The respective quantities are volumetrically defined by the barrel 8 of the liquid A, the sack 9 of the powder B, the box 10 of the granulate C, the flask of the sample D, and the shoe sole E itself.

In a next step S2, a characteristics ruleset for selecting a primary product is set. The characteristics ruleset specifies at least one characteristic value determination rule configured to determine at least on characteristic value based on at least one record, and which specifies at least one record applicable to the characteristics ruleset. For example, a characteristics ruleset for selecting one barrel 8 of liquid A out of many barrels 8 is set. For example, a viscosity below a threshold is needed, and it is known that the viscosity of the liquid A is affected by a maximum temperature of a preceding process. In this exemplary case, there might be specified a rule defining a maximum temperature for a time series of temperatures, and the temperature of the preceding process, as indicated by the corresponding label 7, is set as the record to which the rule is to be applied on. The characteristics ruleset may be set in advance.

In a next step S3, at least one available distinguishable quantity is selected.

In a next step S4, the previously set characteristics ruleset is provided. For example, it may be loaded into a memory, it may be delivered via email from another department, and/or it may be retrieved from a database, including the database 3.

Next is a step S5, wherein sub-steps S6, S7, and S8 are performed at least once, but may be recursively performed. First, in sub-step S6, one data structure 4 held by the database 3 is accessed via the database 3. In the next sub-step S7, at least one report held in a data item 5 of the accessed data structure 4′, which is specified by the characteristics ruleset, is read. Then, in the sub-step S8, at least one characteristic value is determined by applying the at least one read record specified by the characteristics ruleset to at least one rule specified by the characteristics ruleset.

The accessed data structure 4′ may hold further object identifiers, which indicate a primary product to the distinguishable quantity of the (secondary) product indicated by the own object identifier of the accessed data structure 4′. If the accessed data structure 4′ holds such further object identifiers, then the method preferably repeats the sub-steps of S5 for every such further object identifier. Preferably, the additional steps S7 do not result in additional characteristic values, but in a precision gain to the one or more characteristic values determined in the first performing of step S7.

Next, in a step S9, the at least one determined characteristic value is stored by setting a data item 5 of the data structure 4, which data structure 4 is associated to the distinguishable quantity selected in step S3.

A preferred option is to implement a binning ruleset in steps S2 and S4. That is, the characteristic value is merely an indicator of a bin. For example, if the temperature check results in the finding, that the viscosity is in the desired range, then the characteristic value might be one of the following simple examples: “viscosity: good”, “temperature under threshold”, and/or a graphic symbol like a green circle.

In a next step S10, each own object identifier is added to a selection list, which own object identifier is held in a data item 5 of the accessed data structure 4′ if a pre-set criterion is matched by the at least one characteristic value determined in S8.

Then, in a step S11, each data structure 4′, that is holding an own object identifier listed in the selection list, is provided, such as it is made available and loaded. This step may include paying a fee for accessing or using the data structure 4′.

For example, if there are more than one distinguishable quantities available, then a distinguishable quantity of the liquid A from all distinguishable quantities associated to the selection list object identifiers can be chosen based on its age and/or based on a comparison of the ages. Thus, an age indicator indicative of an age of the liquid A is read from the data structure 4′ in a step S12. Further, in step S12, a report may be read from the data structure 4′, which report includes at least one state value indicative of a processing state of a process performed to obtain the distinguishable quantity of the liquid A within the barrel 8.

In a next step S13, for example, an age of the liquid A may be determined. For example, a production date may be read, which is held by a data item 5 of the data structure 4′. Then, in a step S14, one of the distinguishable quantities indicated by the selection list is chosen.

In a next step S11, a distinguishable quantity of a primary product is provided. For example, the barrel 8 of the liquid A is provided, such as it is delivered to a processing device 11, which may be a simple machine but also a processing plant and/or any device in between.

In another example, there is no selection process based on the characteristics ruleset and the age criterion. In this case, the barrel 8 having the distinguishable quantity of the liquid A is provided in step S15. Then, a QR code 12 on the barrel 8 may be scanned. This QR-code 12 may hold the object identifier associated with the distinguishable quantity of liquid A in the barrel 8. Then, in step S11, the data structure 4′ is made available. In another example without the data-base 3, the data structure 4′ itself may be held in the QR-code 12.

Then, step S15 is repeated for the powder B of the sack 9. The powder B has no associated data structure. Rather, it has a lot number, which is printed on a tag 13. Thus, by reading the tag 13, the lot number is provided as a primary product lot identifier in a next step S16.

In a next step, a data owner indicator is provided in step S17. For example, the data owner indicator is selected to be indicative of a company, that is a legal person, owning the processing device 17.

In a next step S18, a process parameter of the processing device 11 may be adjusted based on a report held in the data structure 4′ read in step S12.

In a next step S19, the primary products, here the liquid A and the powder B, are processed to or into a distinguishable quantity of the granulate C. The processing is performed by means of the processing device 11 or by the processing device 11.

It is efficient but not necessary to have a 1:1 ratio of distinguishable quantities. That is, it is possible, for example, to use more than one barrel 8 of the liquid A and less than one sack 9 of the powder B to obtain exactly one distinguishable quantity of the granulate C.

During said processing step S19, sub-steps S20 to S26 may for example be performed.

In a step S20, at least one report of a state value of the processing is generated. Preferably, each state variable or time series of a state variable is reported separately, such that there are preferably one or more reports generated. Each report is generated by a state reporting device 14. Each report includes at least one state value, which is indicative of a processing state during the processing step S19.

In a next step S21, a processing device identifier is recorded, which can be used to distinguish the processing device 11 from other processing devices.

In a next step S22, a state reporting device identifier is recorded. The state reporting device identifier is recorded associated with the report generated by the respective state reporting device. For example in this case, there might be a data item labelled “report 1” holding a report from a first state reporting device 14, a data item labelled “state reporting device 1” holding a serial number of the first state reporting device 14, a data item labelled “report 2” holding a report from a second state reporting device 14, and a data item labelled “state reporting device 2” holding a serial number of the second state reporting device 14. It is not necessary but recommended to record a state recording device identifier for each recorded report.

In a next step S23, a deviation value may be recorded for one or more recorded state values of the one or more recorded state values. A deviation value is indicative of a deviation of the recorded state value from a target value. For example in a case where an exhaustive time series is recorded, some users or applications might only be interested in a summarizing value whereas other users or applications might need the whole time series. Thus, in order to save time later on, the deviation is determined and recorded to serve as a summarizing value.

In a next step S24, a product property value is recorded. For example when a producer of the granulate C does not want to disclose internal knowledge, it is beneficial to record a product property, which follows from one or more state values and/or other sources.

In a next step S25, a deviation value indicative of a deviation of said product property value might be recorded.

Also during the processing step and/or at an end of the processing step, a time or timestamp of the processing or of an end of the processing is recorded in a step S26.

Next, indicators to be stored in a new data structure 4 are determined.

For example, in a step S27, an age indicator for a provided distinguishable quantity of a primary product A, B is determined. For example, the age indicator for the liquid A is determined based on the time read in step S12 and the time recorded in step S26.

In a next step S28, a unique object identifier is generated, such that it is indicative of the distinguishable quantity of granulate C, which distinguishable quantity was obtained by the above processing during the processing step S19.

Then, in step S29, a new data structure 4 is generated. The data structure 4 represents the distinguishable quantity of granulate C obtained during the above processing step S19. In this case, step S29 involves sub-steps S30 to S41.

In a step S30, a data item is set, which holds the object identifier generated in step S28 to be the own object identifier of this new data structure 4, as it is indicative of the distinguishable quantity of granulate C, which is represented by this data structure 4.

In a next step S31, a data item is set, which holds one of the reports generated in step S20. Step S31 is preferably repeated for each report generated.

Then, in a step S32, the age indicator determined in S27 may be set to a data item 5 of the data structure 4, that is associated with the distinguishable quantity of the granulate C, linked with the object identifier indicative of the distinguishable quantity of the primary product A. For example, the data item 5 of the data structure 4 may be labelled “age indicator for product A” and have the age indicator as a content 6.

In a next step S33, a data item is set, which holds a state reporting device identifier generated in step S22. Step S33 is preferably repeated for each state reporting device identifier.

In a next step S34, a data item is set, which holds the primary product lot identifier provided in step S16. Step S34 is preferably repeated for each primary product lot identifier.

In a next step S35, a data item is set, which holds the object identifier indicative of the distinguishable quantity of the primary product, in this case: the liquid A, chosen in step S14. Step S35 is preferably repeated for each object identifier indicative of the distinguishable quantity of the primary product. Alternatively, an object identifier may for example be gained in step S10.

In a next step S36, a data item is set, which holds the processing device identifier recorded in step S21. If the processing in step S19 is performed on a series of processing devices, there may be recorded and set more than one processing device identifiers. S17.

In a next step S38, a data item is set, which holds the production timestamp determined in step S26.

In a next step S39, a data item is set, which holds a deviation value recorded step S23. If there is recorded more than one deviation value, then this step preferably is repeated.

In a next step S40, a data item is set, which holds a product property value recorded in step S24. If there are recorded more than one product property values, then this step preferably is repeated.

In a next step S41, a data item is set, which holds a deviation value recorded in step S25 of the product property value recorded in step S24. If there are recorded more than one a deviation value of a product property values, then this step preferably is repeated.

In a next step S42, the sample D is taken from the distinguishable quantity of granulate C ob-tained in step S19. In a next step S43, a test result is generated, by testing at least one property of the granulate C. An exemplary method for generating a test result, performed in this case includes: performing steps S42 and S43; generating an unique object identifier indicative of the test result in a step S44; and generating a data structure having at least two data items, wherein: one data item holds the object identifier indicative of the test result, one optional data item holds the object identifier indicative of the distinguishable quantity of a product C from which distinguishable quantity the sample was taken, and one further data item holds the generated test result.

That is, in step S44 is generated a test result indicator. Then in a step S45, the method 100 may set a data item of the data structure 4 representing the distinguishable quantity of the granulate C, such that it holds the test result indicator generated in step S44. However, it is preferred to either include the object identifier of the distinguishable quantity of the granulate C in the data structure representing the test sample or to include the test result indicator in the data structure 4 representing the distinguishable quantity of the granulate C.

In a next step S46, a cryptographic representation of the data structure 4′ associated with the liquid A may be determined. This cryptographic representation is then set in a data item 5 of the data structure 4 in a step S47. Thus, it is possible for a customer of the granulate C to reliably reproduce a blockchain-type history of predecessor products, in this case product A, and the S15, S34, and S35.

In next steps S48, S49, and S50 the steps S4, S8, and S9 are repeated. However in this case, the characteristic value is determined for a report generated in step S20 and/or during step S19. Preferably, the characteristics ruleset differs between steps S4 to S9 and steps S48 to S50. That is, this characteristic value represents a characteristic of the granulate C. Thus, computation power can be saved in future, which is beneficial if more than one requests for the characteristic value are to be expected.

In a next step S51, at least one data item 5 of the data structure 4 is encrypted. Thus, confidentiality of data, which is shared not only with a restricted number of parties, can be ensured. It is preferred to have different encryption and de-encryption key sets for different data items. That way access can be granted to selected parameters only.

A preferred combination of steps is steps S48, S49, S50, and S51. This combination has the advantageous effect of graduated confidentiality. For example, all parties get access to the characteristic values, but only few parties get a key for de-encrypting.

In a next step S52, the distinguishable quantity of the granulate C obtained in step S19 is provided. Preferably, the distinguishable quantity is separately provided, which is why it is being rendered distinguishable. Preferably, the distinguishable quantity is provided at an identifiable location. Preferably, said providing of the distinguishable quantity includes a step S53 of packaging said distinguishable quantity of granulates C obtained from said processing into a package. The package in this case is the box 10. Another preferred example of the package is a so-called octabin.

Then, in a next step S54, a data item 5 of the data structure 4 is set, which holds a location identifier indicative of the location, where the distinguishable quantity is provided in above step S52.

In a next preferred step, the separated distinguishable quantity of the granulate C obtained from step S19 is linked to the data structure 4 generated in step S29. This preferably includes a step S56 of attaching a link object to said box 10. In the example of FIG. 1, said step S56 has a sub-step S57, wherein a link object, which is another QR-code 15 for example, is printed on the box 10. The QR-code 15 preferably includes the own object identifier of the distinguishable quantity of granulate C comprised in the box 10.

It is also possible to attach an identifier device, to a secondary product in a next step S58. For example, if the method was performed for the process phase of producing shoe soles E from granulate C, an RFID tag 16 may be implemented into the shoe sole E. The RFID tag 16 is one example for an identifier indicative of an associated own object identifier (of the shoe sole E in this case). Thus, the properties of the shoe sole E can be downloaded by a recycling device 17 during recycling of the shoe sole E.

In a next step S59, a cryptographic representation of the data structure 4 is determined, before in a step S60, the data structure is signed. However, there are methods where the steps S59, S60 are performed in reversed order. The cryptographic representation of the signed data structure 4 is then set into a data item 5 in a step S61. Thus, it is possible to reliably attribute the contents 6 of the data structure 4 to a signature.

Preferably after signing, a distinguishable quantity owner indicator is provided in step S62 and is set to a data item 5 in a step S63. In most cases, an ownership of the distinguishable quantity of granulate C will change, for example when the granulate C is sold from a producer of granulates to a producer of shoe soles. Thus, the change of ownership can be documented at the single instance, which is the database 3.

It is to be noted here that after a change of ownership has been set to the associated data item, preferably the steps S46 and S47 are repeated for the sake of increased reliability and trust.

Then in a step S64, a status indicator is generated. The status indicator is indicative of a current status of a variable factor of the distinguishable quantity of the granulate C. Next, a data item 5 of the data structure 4 associated with the distinguishable quantity of the granulate C is set in a step S65, such that it holds said status indicator. The status indicator may indicate, for example, whether the distinguishable quantity of the granulate C can be bought, whether it has been produced yet, whether it still exists or is used up, and many more. That is, the status indicator makes it possible to perform logistical tasks in addition to technical tasks with the same data-base 3. Thus, consistency is improved and a data storage for redundant data can be reduced.

Then, the method 100 ends with a step S66, where the data structure associated with the liquid A is added to and/or updated in the database 3, and a step S67, where the data structure associated with the granulate C is added to and/or updated in the database 3.

### REFERENCE SIGNS

2 server
3 database
4 data structure
4′ data structure
5 data item
6 content of data item
7 label of data item
8 barrel
9 sack
10 box
11 processing device
12 QR-code
13 tag
14 state reporting device
15 QR-code
16 RFID tag
17 recycling device
100 method
A liquid
B powder
C granulate
D sample
E shoe sole
S1 providing access to a database
S2 setting a characteristics ruleset for selecting a primary product
S3 selecting at least one quantity
S4 providing the pre-set characteristics ruleset
S5 performing in a recursive manner
S6 accessing via the database one data structure
S7 reading at least one report held in an data item
S8 determining at least one characteristic value
S9 setting a data item holding a determined characteristic value
S10 adding to a selection list an own object identifier
S11 providing a data structure associated with the primary product
S12 reading from the data structure associated with the primary product
S13 determining an age for each distinguishable quantity of a primary product
S14 choosing a distinguishable quantity of a primary product based on its age
S15 providing a distinguishable quantity of a primary product
S16 providing a primary product Iot identifier
S17 providing a data owner indicator
S18 adjusting a process parameter
S19 processing the distinguishable quantity of the primary product into a distinguishable quantity of a secondary product
S20 generating a report including a state value
S21 recording a processing device identifier
S22 recording a state reporting device identifier
S23 recording a deviation value of the recorded state value
S24 recording a product property value
S25 recording a deviation value of the recorded product property value
S26 determining a production timestamp
S27 determining an age indicator for a distinguishable quantity of a primary product
S28 generating an unique object identifier
S29 generating a new data structure
S30 setting a data item holding an own object identifier
S31 setting a data item holding a report
S32 setting a data item holding an age indicator for a distinguishable quantity of a primary product
S33 setting a data item holding a state reporting device identifier
S34 setting a data item holding a primary product lot indicator
S35 setting a data item holding an object identifier indicative of a distinguishable quantity of a primary product
S36 setting a data item holding a processing device identifier
S37 setting a data item holding a data owner indicator
S38 setting a data item holding a production timestamp
S39 setting a data item holding a deviation value of the recorded state value
S40 setting a data item holding a product property value
S41 setting a data item holding a deviation value of the recorded product property value
S42 taking a sample
S43 generating a test result
S44 generating a test result indicator
S45 setting a data item holding a test result indicator
S46 determining a cryptographic representation of a data structure associated with a primary product
S47 setting a data item holding a cryptographic representation of a data structure associated with a primary product
S48 providing a characteristics ruleset
S49 determining at least one characteristic value
S50 setting a data item holding a characteristic value
S51 encrypting at least one data item
S52 separately providing a distinguishable quantity at an identifiable location
S53 packaging the distinguishable quantity into a package
S54 setting a data item holding a location identifier
S55 linking the separated distinguishable quantity to the generated data structure
S56 attaching a link object to said package
S57 printing the link object on the package
S58 attaching an identifier device to a secondary product
S59 determining a cryptographic representation of the data structure
S60 signing the data structure
S61 setting a data item holding a cryptographic representation of the signed data structure
S62 providing a distinguishable quantity owner indicator
S63 setting a data item holding a distinguishable quantity owner indicator
S64 generating a status indicator
S65 setting a data item holding a status indicator
S66 adding into a database a data structure associated with the primary product
S67 adding into a database a data structure associated with the secondary product

1. A method (100) for processing at least one primary product (A, B) into at least one secondary product (C), comprising:
providing (S15) at least one distinguishable quantity of a primary product (A, B);
providing (S11), for at least one provided distinguishable quantity of a primary product (A), a data structure (4′) associated with the distinguishable quantity, wherein the data structure (4′) comprises one data item (5) holding an object identifier indicative of the distinguishable quantity of the primary product (A), and one further data item (5) holding a report comprising at least one state value indicative of a processing state of a process performed during obtaining the primary product (A) of the distinguishable quantity indicated by the object identifier;
processing (S19) the at least one primary product (A, B) into a distinguishable quantity of a secondary product (C) by a processing device (11);
generating (S28) an unique object identifier indicative of the distinguishable quantity obtained by the processing during the processing step (S19);
generating (S29) a new data structure (4) having at least two data items (5), wherein one data item (5) holds the generated object identifier indicative of the distinguishable quantity of the secondary product (C), and wherein one further data item (5) holds the object identifier indicative of the distinguishable quantity of the primary product (A); and
providing (S52) the distinguishable quantity obtained by the processing during the processing step (S19).

2. The method according to any preceding claim, wherein during the providing (52) the distinguishable quantity is separately provided, and wherein the method further comprises:
linking (S55) the separated distinguishable quantity to the generated data structure (4).

3. The method according to claim 1, wherein, during the processing step (S19), the at least one primary product (A, B) is processed into multiple distinguishable quantities of the same secondary product (C) and/or of different secondary products (C), wherein at least the steps: generating (S28) a unique object identifier, generating (S29) a data structure, providing (S52) the distinguishable quantity, and, if applicable, the steps: linking (S55) the separated distinguishable quantity and attaching (S56) the link object, are performed for each of multiple of the distinguishable quantities.

4. The method according to claim 1, further comprising:
providing (S16) a lot of a primary product (B) and a primary product lot identifier indicative of the lot of the primary product (B); and
setting (S34) a data item (5) of the data structure (4) such that it holds the primary product lot indicator.

5. The method according to claim 1, further comprising:
providing (S1) access to a database (3), that is configured to include multiple data structures (4, 4′) each having at least two data items (5) and is configured to provide access to a data structure (4, 4′) such that a data item (5) is addable the accessed data structure (4, 4′), is amendable within the accessed data structure (4, 4′) and is removeable from the accessed data structure(4, 4′); and
adding (S67) into the database (3) the data structure (4) associated with the distinguishable quantity of the secondary product (C).

6. The method according to claim 1, further comprising:
providing (S1) access to a database (3), that is configured to include multiple data structures (4, 4′);
accessing (S6) via the database (3) one data structure (4) held by the database (3); and
adding a data item to the accessed data structure (4, 4′), amending (S9) a data item (5) of the accessed data structure (4) and/or removing a data item (5) from the accessed data structure (4, 4′).

7. The method according to claim 1, further comprising:
taking (S42) a sample from the distinguishable quantity of the secondary product (C); generating (S43) a test result indicative of at least one material property of the product (C) of the processed and/or separated distinguishable quantity; generating (S44) a test result indicator indicative of the generated test result; and setting (S45) a data item (5) of the data structure (4) such that it holds the test result indicator.

8. The method according to claim 1, further comprising:
signing (S60) the data structure (4) by setting a data item (5) of the data structure (4) such that it holds a signature indicative of natural or legal person and/or a group thereof; and
setting (S61) a data item (5) of the data structure (4) such that it holds a cryptographic representation of the signed data structure (4).

9. The method according to claim 1, further comprising:
determining (S46) a cryptographic representation of the data structure (4′) associated with the distinguishable quantity of the primary product (A); and
setting (S47) a data item (5) of the data structure associated with the secondary product (C), such that it (5) holds the cryptographic representation of the data structure associated with the distinguishable quantity of the primary product (A).

10. The method according to claim 1, wherein the primary product (A, B) is a chemical product or a chemical raw material, and the secondary product (C) is a chemical product.

11. The method according to claim 1, wherein a data item of the primary data structure holds a production timestamp indicative of a time when the primary product was produced, and
wherein the method further comprises:
determining (S27) an age indicator indicative of a duration from the time indicated by the production timestamp until a time when the primary product (A) is provided; and
setting (S32) a data item (5) of the data structure (4) associated with the distinguishable quantity of the secondary product (C) such that it holds the age indicator linked with the object identifier indicative of the distinguishable quantity of the primary product (A).

12. A non-transitory computer-readable medium storing thereon instructions, wherein the instructions, when executed by at least one processor unit, cause the at least one processor unit to perform operations including the method according to claim 1.

13. A non-transitory computer-readable medium having stored thereon a data structure (4) having at least two data items (5), wherein:
one data item (5) holds a second object identifier, that is configured to be a unique object identifier for identifying a processed distinguishable quantity of a product; and
at least one data item (5) holds a first object identifier, that is configured to be a unique object identifier for identifying a primary distinguishable quantity of a primary product (A, B) processed to a distinguishable quantity identified by the second object identifier.

14. The non-transitory computer-readable medium of claim 13, wherein the data structure comprises at least one of the following data items (5): a data item (5) holding a at least one report, wherein each report is configured to have at least one state value indicative of a processing state of a step of processing (S19) a primary product (A, B) of the distinguishable quantity indicated by the first object identifier into the distinguishable quantity of the product (C) indicated by the second identifier; a data item (5) holding a package identifier indicative of the at least one package having the distinguishable quantity indicated by the object identifier; a data item (5) holding a location identifier indicative of an identifiable location of the distinguishable quantity indicated by the object identifier; a data item (5) holding a data owner indicator indicative of a natural or legal person and/or a group thereof owning this data structure; a data item (5) holding a distinguishable quantity owner indicator indicative of a natural or legal person and/or a group thereof owning the distinguishable quantity indicated by the object identifier; a data item (5) holding a primary product lot identifier indicative of a lot of the primary product (A, B); and/or a data item (5) holding a test result indicator indicative of the generated test result based on a sample taken from the distinguishable quantity indicated by the object identifier.

15. (canceled)
